(19) United States
(12) Patent Application Publication
(10) Pub. No.: US 2007/0009560 A1
(43) Pub. Date: Jan. 11, 2007

(54) Cosmetic and dermatological agent containing magnetic particles production thereof and use of the same

(75) Inventors: Karin Golz-Berner, quai Jean Charles Rey (MC); Leonhard Zastrow, avenue des Papalins (MC)

Correspondence Address: AKERMAN SENTERFITT, P.O. BOX 3188, WEST PALM BEACH, FL, 33402-3188, US

(73) Assignee: Coty B.V., Haarlem (NL)

(21) Appl. No.: 10/574,069

(22) PCT Filed: Sep. 30, 2003
(86) PCT No.: PCT/EP03/10847

§ 371 Date: Sep. 11, 2006

(51) Int. Cl. A61K 8/19

(52) U.S. Cl. 424/401; 977/926

(57) ABSTRACT

The invention relates to a cosmetic and dermatological agent containing magnetically hard particles and other active ingredients. Said agent contains 0.0001 to 2 wt. % of magnetically hard particles, preferably barium hexaferrite single crystals of between 80 and 550 nm and with a coercive force ranging from 80,000 to 1,600,000 A/m; and 0.0001 to 0.05 wt. % of a ground jade stone of between 50 and 95 nm, and cosmetic or dermatological auxiliary and carrier substances up to 100 wt. %. The invention also relates to a method for producing said agent, and to the use thereof for increasing microcirculation, the local regulation of microcirculation, and the immune defence in the skin.

### CROSS REFERENCE TO RELATED APPLICATION

This application is a national stage of PCT/EP2003/010847 filed Sep. 30, 2003.

### BACKGROUND OF THE INVENTION

1. Field of the Invention

The invention relates to a cosmetic and dermatological agent containing magnetically hard particles and other active ingredients as well as to a method for producing said agent and to the use thereof.

2. Description of Related Art

From DE-C-4325071, a preparation for stimulating blood circulation is known, which contains magnetically hard single-domain particles having a coercive field strength of between 3,000 and 5,000 oerstedts and grain sizes ranging from 600 to 1,200 nm in addition to cosmetic or pharmaceutical carrier substances. Single-domain particles mentioned in said publication are e.g. barium and strontium hexaferrites, which on the whole stimulate blood circulation.

Further, it is known from WO 98/44895 that wounds will heal even better if magnetically hard particles sized 100-550 nm are used in combination with asymmetric lamellar aggregates which consist of phospholipids and fluorocarbons and are loaded with oxygen.

Moreover, EP 236374 discloses the use of cosmetic compositions containing powdered precious stones whose grain size ranges between 0.5 and 30 μm as colorant with low allergenic potential.

### SUMMARY OF THE INVENTION

The object of the invention is to improve blood circulation in the skin and the immune defence.

According to the invention, a cosmetic and dermatological agent is provided with a share of magnetic particles and which agent comprises

0.001 to 2 wt. % of magnetically hard particles, selected from the group consisting of barium hexaferrite single crystals, strontium hexaferrite single crystals, samarium-cobalt particles (SmCo) and neodymium-iron-boron particles (Nd₂Fe₁₄B),

the particle size ranging between 80 and 550 nm in each case and the particles' coercive force ranging from 80,000 to 1,600,000 A/m;

0.0001 to 0.05 wt. % of a ground jade stone substantially consisting of jadeite, nephrite or a mixture thereof

the particle size of which ranges between 50 and 95 nm;

and cosmetic or dermatological auxiliary, carrier or other active substances or a mixture thereof up to 100 wt. %.

In one embodiment of the invention, the magnetically hard particles are encapsulated in aqueous liposomes consisting of phospholipids and enclosed in a gel.

In another advantageous embodiment, the magnetically hard particles can also be incorporated in asymmetric lamellar aggregates, which asymmetric lamellar aggregates consist of natural phospholipids such as lecithin and fluorocarbons and in addition contain alcohols, water and a gel-forming substance. The jade particles can also be incorporated in the aforesaid “complex.”

In yet another embodiment of the invention, asymmetric lamellar aggregates can be present in addition to the aforesaid liposomes with magnetically hard particles or asymmetric lamellar aggregates with magnetically hard particles or mixtures thereof. Such lamellar aggregates consist of natural phospholipids containing phosphatidylcholine in an amount ranging between 10 and 99 wt. %, preferably 30-99 wt. %, and fluorocarbons and are loaded with oxygen up to the saturation limit.

The magnetically hard particles have a particle size ranging between 80 and 550 nm. Small amounts of larger or smaller particles can be contained (below 5%), however, it is essential that the medium particle size D₅₀ be approximately 250 nm. This means that at least 50% of the magnetically hard particles contained in an emulsion measure about 250 μm. A typical particle size distribution is e.g.

15% 80 to 100 nm

55% 100 to 250 nm

30% 250 to 350 nm.

A preferred particle size is therefore 80-350 nm.

The magnetic particles used are single crystals with uniform magnetic orientation. Non-doped barium and strontium hexaferrites are particularly preferred. These single crystals are e.g. produced according to the glass crystallization technique, i.e. by growing them from a quenched glass melt as described in DE 19715477. Suitable glasses are e.g. those consisting of 20-50 wt. % Fe₂O₃, 30-50 wt. % BaO and 20-50 wt. % B₂O₃.

Surprisingly, it has been found that the functional state of microcirculation can be increased to considerably above the value to be expected by adding 0.0001 to 0.05 wt. % of finely ground jade stone to the magnetically hard particles, relative to the comparative value achieved without said addition. In this way, the good effect on microcirculation of the known magnetically hard particles in cosmetic compositions is clearly exceeded, thus improving the supply of oxygen and nutrients and the removal of metabolic end products in skin tissue even further.

At the same time, the local regulation of microcirculation increased to considerably above the value to be expected, relative to the comparative value achieved without said addition. The good values achieved by the magnetically hard particles alone are again exceeded, thus decisively improving the adaptation width (regulation width) of microcirculation to changing metabolic needs of the skin.

It has further been found that the boundary conditions for the immunological activity of the white blood cells, i.e. the main actors of the body's defence, were improved to an extent which is extraordinarily surprising. Compared to a comparative substance containing only the magnetically hard particles and carrier substances, a cosmetic agent according to the invention with the same concentration of magnetically hard particles to which just 0.00025 wt. % of ground jade stone, relative to the overall composition, had been added increased the immune defence by another 15-30%.

The foregoing provides proof for the functional state of microcirculation, the regulation width thereof and the state of the immune defence for a cosmetic and dermatological product, which has not been achieved so far for the prophylactic and real anti-ageing effect of other products.

The jade stone is preferably provided as jadeite. A mixture of jadeite and nephrite or nephrite alone can also be used.

In another embodiment of the invention, the jade stone can be mixed with a ground malachite stone of the same grain size in a ratio of 1:0.2-3.5, whereby nearly the same results can be achieved.

The cosmetic or dermatological agent according to the invention further contains usual auxiliary and carrier substances such as e.g. water, preservatives, colourants, pigments having a colouring effect, thickeners, fragrances, alcohols, polyols, esters, electrolytes, gel-forming substances, polar and non-polar oils, polymers, copolymers, emulsifiers, waxes, stabilizers.

Other active ingredients can also be contained. Cosmetic active ingredients include e.g. inorganic and organic sunscreens, free radical scavengers, moisturizers, vitamins, enzymes, plant-based active substances, polymers, antioxidants, anti-inflammatory natural active substances; products obtained by the gentle ultrasonic decomposition of yeasts or vegetable matter according to WO 94/13783.

Pharmacologically active substances which are particularly suitable are heparin, acetylsalicylic acid, piroxicam, miroxicam, oestrogens, pseudohormones or phytohormones or mixtures thereof.

The cosmetic or pharmacologically active substances can be incorporated in the aqueous phase of the emulsion; they can also be enclosed separately in liposomes.

Advantageously, the inventive agent is provided as a gel or an emulsion or a gel emulsion; the latter e.g. with emulsifiers, water and a gel such as Carbopol.

Another embodiment of the invention consists in that 0.1 to 10 wt. % of a cosmetically acceptable solid electret with a particle size ranging between 0.05 and 100 μm is added to the inventive mixture of magnetically hard single crystals and ground jade stones, said electret having an induced permanent dipole moment and a permanent electric field whose field strength ranges between 500 and 10⁷ Vm⁻¹.

Preferred electrets have a field strength of between 10⁵ and 10⁷ Vm⁻¹.

In general, the term electret refers to substances which have a permanent opposite electric charge on two opposing surfaces. They thus create a permanent electric dipole surrounded by an electric field. Certain natural electrets such as tourmaline have been known for a long time. However, a number of substances can also be made electrets by acting on them from the outside. This can be done by means of a thermal procedure, i.e. by heating the substance to above the Curie temperature and exposing it to an electric field in this heated state. The dipoles which orient themselves in the field are then frozen by cooling down the substance.

The non-equilibrium state achieved in this way returns to an equilibrium state after a certain relaxation time, which can be several years for electrets. The same states can also be brought about by means of photoelectric procedures.

The dipole moment, which is referred to as “induced permanent electric dipole moment” herein, is clearly higher than the known dipole moment of substances which have not been induced.

Once the electret material has been induced, it is comminuted to a particle size of between 0.05 and 100 μm, preferably 3 and 80 μm, for use in a cosmetic.

By adding these substances, the skin cells' ability to absorb nutrients and active substances can be clearly enhanced, thus increasing the main activity of the inventive agents even further.

Preferred electret materials are polymerized fluorocarbons, selected from the group consisting of polytetrafluoroethylene (PTFE), fluoroethylenepropylene (FEP), polyvinylidene fluoride (PVDF), amorphous fluoropolymer (AF) and ground tourmaline as natural electret as well as mixtures thereof. PTFE is particularly preferred.

The invention also relates to a method for producing the cosmetic or dermatological agent containing magnetic particles. Said method consists in that at first a mixture of magnetically hard particles, jade particles, one or several fluorocarbon(s), one or several phospholipid(s), water, one or several monovalent and polyvalent alcohols and a gel-forming substance is prepared, and the gel obtained is mixed with further cosmetic auxiliary, carrier or active substances or mixtures thereof at a temperature ranging between 28 and 42° C., without increasing the temperature to above 42° C.

Said jade particles preferably consist of jadeite.

Phospholipids which can be used include e.g. phosphatidylcholine, phosphatidyl ethanolamine, phosphatidylinositol, phosphatidyl serine, phosphatidic acid and lysolecithins as well as mixtures thereof. Known products are e.g. Phoslipon® or NAT®. However, equivalent substances, such as sphingolipids, e.g. sphingomyelins or ceramides, can also be used.

Preferred monovalent alcohols are ethanol and isopropanol. Polyvalent alcohols which can be used include e.g. propylene glycol, dipropylene glycol, ethylene glycol, isoprene glycol, glycerine, butylene glycols, sorbitol and mixtures thereof. Glycerine, propylene glycol and mixtures thereof are preferred.

Suitable gel-forming substances include carbomer, xanthan gum, carrageenan, acacia gum, guar gum, agar-agar, alginates and tyloses, carboxymethyl cellulose, hydroxyethyl cellulose, quaternized cellulose, quaternized guar, certain polyacrylates, polyvinyl alcohol, polyvinylpyrrolidone, montmorillonite.

The aforesaid mixture, which is prepared separately according to the invention and which is also referred to as “complex” herein, can contain further substances such as preservatives, pH adjusters, e.g. triethanolamine, etc.

The invention also relates to the topical cosmetic use of a mixture of magnetically hard single crystals of barium hexaferrite or strontium hexaferrite with a particle size of 50-550 nm, said single crystals making up 0.001-2 wt. %, together with ground jade stone with a particle size of 30-95 nm, jade making up 0.0001 to 0.05 wt. %, and cosmetic auxiliary substances for increasing microcirculation, the local regulation of microcirculation and the immune defence to values which are at least 15% above those achieved by a comparative preparation containing the same amount of magnetically hard single crystals alone.

Moreover, the invention relates to the topical dermatological use of a mixture of magnetically hard single crystals of barium hexaferrite or strontium hexaferrite with a particle size of 50-550 nm, said single crystals making up >2 to 6 wt. %, together with ground jade stone with a particle size of 30-95 nm, jade making up 0.05 to 3 wt. %, and dermatological auxiliary substances for producing a preparation for increasing microcirculation, the local regulation of microcirculation and the immune defence to values which are at least 20% above those achieved by a comparative preparation containing the same amount of magnetically hard single crystals alone.

All percentages are relative to the weight of the overall composition.

### BRIEF DESCRIPTION OF THE DRAWINGS

The invention will now be explained in more detail by means of examples. In the attached drawing,

FIG. 1: shows a diagram illustrating the distribution of red blood cells with regard to microcirculation in the skin;

FIG. 2: shows a diagram illustrating the skin's immunological resistance.

### DETAILED DESCRIPTION OF THE INVENTION

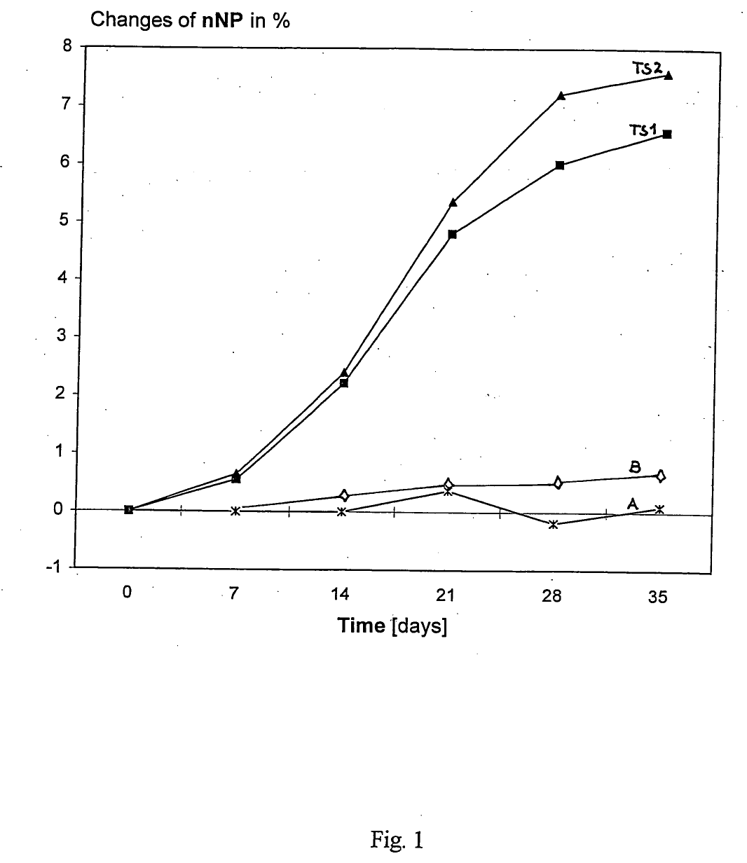

FIG. 1 shows the distribution of red blood cells over time in per cent, based on the measured number of nodal points which are perfused with blood cells at each moment [nNP]; starting value n=60. Said nodal points which are perfused with blood cells are the branch points in the microvessel network and reflect the flow of blood cells through the capillaries. The homogeneous group of test persons selected at random (18 female test persons aged 35-45 years, normal skin type) was studied over 35 days, carrying out double blank tests every 7 days. For this purpose, an intravitalmicroscopic test unit (OLYMPUS, Japan; KONTRON, USA) and a reflectance spectrometric test unit (SPEX, USA) were used. The biometric analysis of the data obtained was done using the WILCOXON rank sum test at significance level α=5% (two-sided test).

FIG. 1 shows that both test substances TS1 and TS2 improve the distribution condition of blood with regard to the skin's microcirculation to a biologically relevant extent. Significant differences in characteristics can be observed from the 14th day at the latest (WILCOXON rank sum test). Test substance TS2 shows an increase which is clearly above the additive increase to be expected, i.e. there is a synergy if the magnetically hard particles are combined with jade particles.

The venular flow out of the networks [Qven], which is not shown in a diagram herein, shows an analogous behaviour as regards characteristics, which is quite important since most microcirculatory disorders and consequently most therapeutic concepts concern the venular flow. An analogous behaviour can also be observed in respect of the local regulation width of microcirculation, specifically for the characteristic “surface area below the envelope of the amplitude-frequency spectrum [AVM]”.

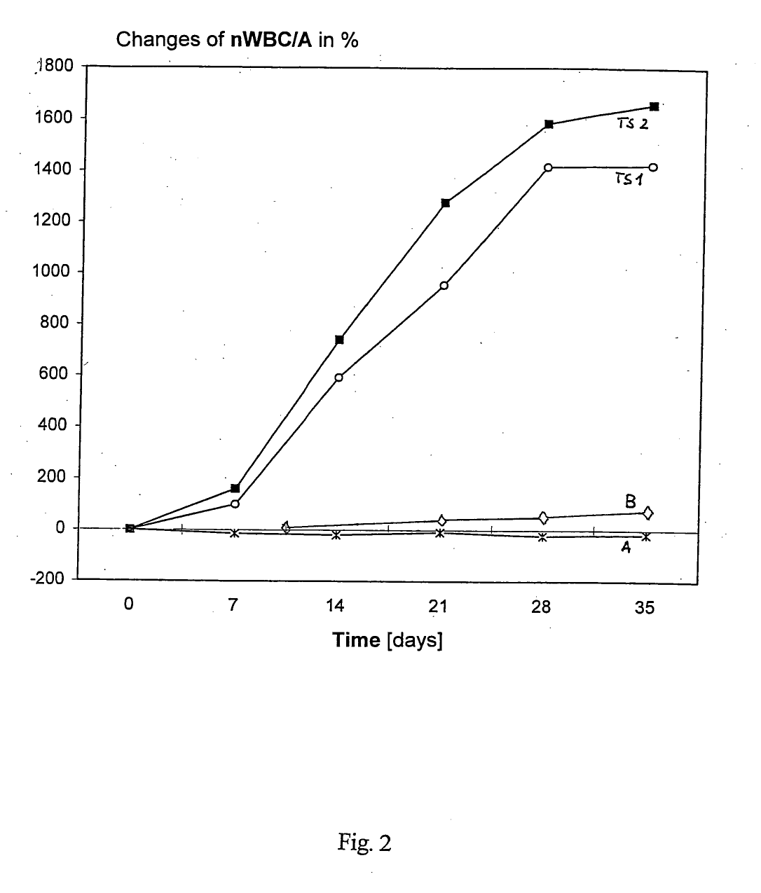

FIG. 2 shows the immunological resistance of the skin in per cent, based on the measured number of white blood cells adhering to a defined venule wall [nWBC/A]. The increase over time shows that the microhemodynamic boundary conditions for the adhesion of white blood cells to the venule endothelium are improved and consequently there are more and more adhesions. The test conditions were the same as mentioned above with reference to FIG. 1, and the tests were carried out during the same period of time on different surface areas of the skin of the female test persons' left forearms.

FIG. 2 shows that both test substances TS1 and TS2 improve the skin's immunological resistance to a biologically relevant extent, wherein significant differences as regards characteristics can be observed from the 14th day onwards (according to WLCOXON rank sum test). As in FIG. 1, there is a synergistic activity.

The test substance TS1 corresponds to the active complex according to Example 1 without jade addition, TS2 to the same complex with jade addition. Curve A is the control value of a non-treated skin area, and curve B is the value for the active complex without magnetic particles, but with jade particles.

Compared to known products, the inventive agents increase microcirculation, the regulation width thereof and the state of the immune defence to an extent which could not be expected, as has been demonstrated by the aforedescribed tests.

The following quantities in the examples are in weight per cent unless indicated otherwise.

EXAMPLE 1
Day cream

| Phase A | |
|---|---|
| Water | q.s. ad 100 |
| Glycerine | 3 |
| Crosspolymer | 0.25 |
| Phase B | |
| Steareth-21 | 2.1 |
| Steareth-2 | 1.35 |
| Stearyl Alcohol | 0.5 |
| Dimethicone | 4.0 |
| Macadamia Seed Oil | 2.0 |
| Phase C | |
| Triethanolamine (TEA) | 0.25 |
| Phase D | |
| Active complex* | 0.05 |
| Perfume | 0.5 |
| Preservative | 0.3 |

*6% barium hexaferrite particles D₅₀ = 230 nm, 160,000-280,000 A/m; 0.5% jade particles D₅₀ = 75 nm; 2% perfluorodecalin; 5% phospholipids; 2% propylene glycol; 10% glycerine; 1% carbomer; 3% ethanol; 0.1% TEA; 70.4% water.

Phases A and B were prepared separately and stirred into one another and mixed at 70° C. Phase C was added at 40° C., and Phase D was prepared separately and stirred into the mixture at 35° C.

EXAMPLE 2
Night cream

| Water | q.s. ad 100 |
|---|---|
| Glycerine | 3 |
| Crosspolymer | 1.5 |
| Phase B | |
| Shea Butter Fruit | 10 |
| Petrolatum | 4 |
| Silicone | 5.0 |
| Phase C | |
| Triethanolamine (TEA) | 1.5 |
| Phase D | |
| Active complex* | 0.5 |
| Perfume | 0.5 |
| Preservative | 0.5 |

*As in Example 1, except that 25% barium hexaferrite particles and 1% jade particles are contained.

EXAMPLE 3
Cosmetic mask

| Phase A | |
|---|---|
| Water | q.s. ad 100 |
| Glycerine | 5.0 |
| Crosspolymer | 1.0 |
| Phase B | |
| Petrolatum | 10.0 |
| Silicone | 8.0 |
| Phase C | |
| TEA | 1.0 |
| Phase D | |
| Active complex* | 0.8 |
| Teflon ® (PTFE) | 1.5 |
| Perfume | 0.5 |
| Preservative | 0.5 |

*As in Example 1, except that 1% barium hexaferrite particles and 0.5% jade particles are contained; water 75.4%.

1-10. (canceled)

11. A cosmetic and dermatological agent containing magnetic particles comprising 0.0001 to 2 wt. % of magnetically hard particles, selected from the group consisting of barium hexaferrite single crystals, strontium hexaferrite single crystals, samarium-cobalt particles (SmCo) and neodymium-iron-boron particles (Nd₂Fe₁₄B), the particle size ranging between 80 and 550 nm in each case and the particles' coercive force ranging from 80,000 to 1,600,000 A/m; and 0.0001 to 0.05 wt. % of a ground jade stone the particle size of which ranges between 50 and 95 nm;
and cosmetic or dermatological auxiliary and carrier substances up to 100 wt. %.

12. An agent according to claim 11, wherein said magnetically hard particles are incorporated in liposomes, asymmetric lamellar aggregates or mixtures thereof in a gel.

13. An agent according to claim 11, wherein said agent additionally contains asymmetric lamellar aggregates loaded with oxygen.

14. An agent according to claim 11, wherein the share of jade stone is in the rage of 0.002 to 0.02 wt. %.

15. An agent according to claim 1 1, wherein said agent further contains 0.1 to 10 wt. % of a cosmetically acceptable solid electret with a particle size ranging between 0.05 and 100 μm, said electret having an induced permanent dipole moment and a permanent electric field whose field strength ranges between 500 and 10⁷ Vm⁻¹.

16. An agent according to claim 15, wherein said electret is polytetrafluoroethylene (PTFE), fluoroethylenepropylene, polyvinylidene fluoride, amorphous fluoropolymer, tourmaline or a mixture thereof, preferably PTFE.

17. An agent according to claim 11, wherein said agent contains a mixture of jade and malachite.

18. A method for producing a cosmetic or dermatological agent containing magnetic particles according to claim 11, wherein a mixture of magentically hard particles, jade particles, one or several fluorocarbon(s), one or several phospholipid(s), water, one or serveral monovalent and polyvalent alcohols and a gel-forming substance is prepared, and the gel obtained is mixed with further cosmetic auxiliary, carrier or active substances or mixtures thereof at a temperature ranging between 28 and 42° C., without increasing the temperature above 42° C.

19. The topical cosmetic use of a mixture of magnetically hard single crystals of barium hexaferrite or strontium hexaferrite with a particle size of 50-550 nm, said single crystals making up 0.001 to 2 wt. %, together with ground jade stone with a particle size of 30-95 nm, jade making up 0.0001 to 0.05 wt. %, and cosmetic auxiliary substances for increasing microcirculation, the local regulation of microcirculation and the immune defence to values which are at least 15% above those achieved by a comparative preparation containing the same amount of magnetically hard single crystals alone.

20. The topical dermatological use of a mixture of magnetically hard single crystals of barium hexaferrite or strontium hexaferrite with a particle size of 50-550 nm, said single crystals making up >2 to 6 wt. %, together with ground jade with a particle size of 30-95 nm, jade making up 0.05 to 3 wt. %, and dermatological auxiliary substances for producing a preparation to values which are at least 20% above those achieved by a comparative preparation containing the same amount of magnetically hard single crystals alone.
